(19) United States
(12) Patent Application Publication
(10) Pub. No.: US 2026/0314754 A1
(43) Pub. Date: Oct. 15, 2026

(54) DETACHABLE SOLE ON SHOES

(71) Applicant: Choonghwan KIM, Seoul (KR)

(72) Inventor: Choonghwan KIM, Seoul (KR)

(21) Appl. No.: 19/700,521

(22) Filed: Jun. 8, 2026

Related U.S. Application Data
(63) Continuation of application No. PCT/KR2025/007828, filed on Jun. 9, 2025.

(30) Foreign Application Priority Data
Dec. 18, 2024 (KR) 10-2024-0189461

(51) Int. Cl. A43B 13/36; A43B 13/14; A61F 5/058

(52) U.S. Cl. CPC A43B 13/36; A43B 13/145; A61F 5/0585

(57) ABSTRACT

The present disclosure relates to a detachable sole for shoes. The detachable sole according to one embodiment of the present disclosure includes a concave portion formed in an upper surface and configured such that a midsole of the shoe is inserted thereinto, a protruding portion formed to protrude backward from a heel of the shoe, wherein the protruding portion corresponds to the heel of the shoe when the sole is mounted on the shoe, and at least one groove portion formed to have a concave portion facing upward in a bottom surface of the sole.

### CROSS-REFERENCE TO RELATED APPLICATIONS

This application is a continuation of International Application No. PCT/KR2025/007828 filed on Jun. 9, 2025, which claims priority to Korean Patent Application No. 10-2024-0189461 filed on Dec. 18, 2024, the entire contents of which are herein incorporated by reference.

### TECHNICAL FIELD

The present disclosure relates to a sole for shoes, and more particularly to a detachable sole on shoes.

### BACKGROUND

There is known a splint-type shoe (so-called leg protector) which may be used by a patient with foot or leg injury. It is preferable that the patient always wears such a splint-type shoe in various usage environments, such as indoors, outdoors, or the like due to the nature of a splint for fixing the foot or leg.

To do this, a sole having a detachable structure with respect to a splint-type shoe is known (see Patent Document 1). The sole disclosed in Patent Document 1 may be attached to or detached from the shoe depending on a usage environment. Thus, a patient may move around while always wearing the splint-type shoe without taking it off regardless of a change in the usage environment. For example, the patient may move outdoors while mounting the sole to his/her shoe, and may move indoors while separating only the sole from the shoe without taking the shoe off.

Meanwhile, in order to separate the sole from the splint-type shoe in the related art, the patient needs to press the sole with his/her hand(s) and take out his/her foot from the shoe. However, the patient who wears the splint-type shoe cannot bend his/her leg or may have a difficulty performing such an action. In addition, in a case in which the sole is attached to or detached from the shoe in this way, the patient needs to directly handle the shoe or the sole with his/her hand(s), which may cause hygiene issues.

### PRIOR ART DOCUMENT

### Patent Document

Korean Patent No. 10-1694792 (registered on Jan. 4, 2017)

### SUMMARY

The present disclosure is made to solve the above-described matters in the related art.

The present disclosure is for the purpose of providing a sole for shoes, which is capable of being hygienically attached to and detached from a shoe with an improved detachability.

Further, the present disclosure is for the purpose of providing a sole for shoes, which is capable of alleviating shocks to a user's foot when walking.

Representative configurations of the present disclosure to achieve the above purposes are described as follows.

A detachable sole on shoes according to one embodiment of the present disclosure includes: a concave portion formed in an upper surface and configured such that a midsole of a shoe is inserted thereinto; a protruding portion formed at a portion corresponding to a heel of the shoe when the detachable sole is mounted on the shoe and configured to protrude further backward than the heel of the shoe; and at least one groove portion formed to have a concave portion facing upward in a bottom surface of the sole.

According to one embodiment of the present disclosure, the bottom surface may have a round shape along a longitudinal direction when viewed from a lateral side.

According to one embodiment of the present disclosure, the at least one groove portion may be formed to open backward and communicate with an outside in a state in which the bottom surface is brought into contact with a ground.

According to one embodiment of the present disclosure, an inner sidewall of the concave portion may include a coupling portion configured to be coupled to an outer circumferential surface of the midsole.

According to one embodiment of the present disclosure, the coupling portion may include a plurality of coupling holes formed along a periphery of the inner sidewall of the concave portion, the plurality of coupling holes being configured to be coupled to a plurality of coupling protrusions formed on an outer circumferential surface of the midsole, respectively.

According to one embodiment of the present disclosure, an upper surface of the concave portion may include bent portions formed along a longitudinal direction.

A shoe according to another embodiment of the present disclosure includes: a protection part formed to surround at least a portion of a leg of a user; a bottom plate part connected to the protection part and formed to support a foot of the user; a midsole provided below the bottom plate part; and a sole configured to be attachable to and detachable from the midsole. The sole includes: a concave portion formed in an upper surface of the sole and configured such that the midsole is inserted thereinto; a protruding portion formed to protrude backward from a heel of the shoe, wherein the protruding portion corresponds to the heel of the shoe when the sole is mounted on the shoe; and at least one groove portion formed to have a concave shape facing upward in a bottom surface of the sole.

Further, the sole and the shoe including the same according to the present disclosure may further include other additional configurations without departing from the technical sprit of the present disclosure.

According to one embodiment of the present disclosure, a sole mounted on a shoe may be separated from the shoe by pressing a protruding portion of the sole with the other foot. Therefore, a patient with a limited mobility may easily separate the sole from the shoe. Further, the patient does not need to use his/her hand(s) to separate the sole from the shoe. This allows for hygienic usage of the sole.

Further, according to one embodiment of the present disclosure, by using a rounded bottom surface of the sole, it is possible to reduce shocks applied to user's legs when walking.

### BRIEF DESCRIPTION OF THE DRAWINGS

FIGS. 1A and 1B exemplarily illustrate a structure in which a shoe and a sole are coupled to each other according to one embodiment of the present disclosure.

FIG. 2 shows a sole according to one embodiment of the present disclosure.

FIG. 3 shows a bottom surface of a sole according to one embodiment of the present disclosure.

FIG. 4 shows a vertical cross-section of a sole according to one embodiment of the present disclosure.

FIG. 5 shows a state in which a sole according to one embodiment of the present disclosure is coupled to a shoe.

FIG. 6 shows a state in which a sole according to one embodiment of the present disclosure is separated from a shoe.

FIGS. 7A to 7F illustrate walking states when a user walks with a sole according to Example of the present disclosure and a sole according to Comparative example.

FIGS. 8A to 8C are graphs illustrating walking simulation results when the user walks using a sole according to Example of the present disclosure and a sole according to Comparative example.

### Explanation of Reference Numerals

100: Sole
110: Concave portion
120: Protruding portion
130: Doorsill
140: Coupling hole
200: Shoe
210: Protection part
220: Bottom plate part
230: Midsole
231: Coupling protrusion
240: Toecap

### DETAILED DESCRIPTION

Hereinafter, exemplary embodiments of the present disclosure will be described in detail with reference to the accompanying drawings. Here, in the description of the present disclosure, when it is determined that a detailed description of a related known function or configuration may unnecessarily obscure the gist of the present disclosure, the detailed description thereof will be omitted.

The terms used herein are merely used to describe specific exemplary embodiments and do not limit the present disclosure. For example, expressions in the singular form should be understood to encompass expressions in the plural form unless the context clearly indicates otherwise. The term “includes,” “has” or the like used herein are intended to include features, numeric characters, operations, acts, constituent elements, parts, or a combination thereof, and should be understood not to exclude one or more other features, numeric characters, operations, acts, constituent elements, parts, or a combination thereof, or additional features and the like.

Unless otherwise defined, all technical and scientific terms used herein have the same meaning commonly understood by those skilled in the art to which the present disclosure pertains. In addition, the commonly-used predefined terms should be interpreted as consistent with the meanings of the context in the related art and should not be interpreted as having ideal or excessive formal meanings unless otherwise defined in this application.

The same or similar reference numerals will be given to the same constituent elements throughout the specification. Further, a size, thickness, position or the like of each constituent element illustrated in the drawings are arbitrarily illustrated on a large scale for the sake of convenience in description, and hence the present disclosure is not necessarily limited to the shape and size illustrated. That is, it is to be understood that specific shapes, structures, and characteristics described herein may be modified from one embodiment to another embodiment without departing from the spirit and scope of the present disclosure. Positions or arrangements of individual constituent elements may also be modified without departing from the spirit and scope of the present disclosure.

Hereinafter, exemplary embodiments of the present disclosure will be described in detail with reference to the accompanying drawings.

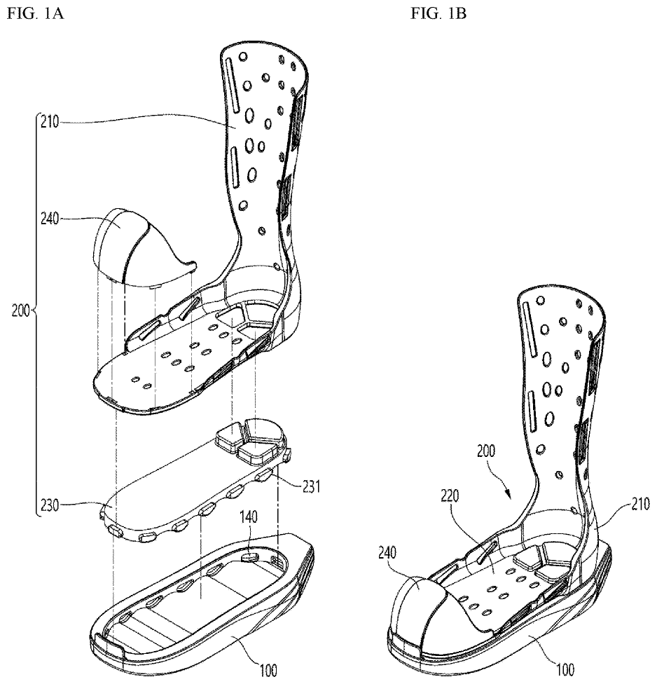

FIGS. 1A and 1B exemplarily illustrate a structure in which a shoe and a sole are coupled to each other according to one embodiment of the present disclosure.

Referring to FIGS. 1A and 1B, a shoe 200 according to one embodiment of the present disclosure is a splint-type shoe and may include a protection part 210, a bottom plate part 220, a midsole 230, and a toecap 240. In the embodiment illustrated in FIGS. 1A and 1B, the shoe 200 is exemplified as the splint-type shoe, a so-called leg protector, but the present disclosure is not limited thereto. The sole 100 according to one embodiment of the present disclosure may be applied not only to the leg protector but also to various types of shoes.

The protection part 210 of the shoe 200 according to one embodiment of the present disclosure may perform a function of fixing and protecting a user's leg. To do this, the protection part 210 has a shape that surrounds at least a portion of the leg, for example, at least a portion of a lateral side or a back side of the leg.

The bottom plate part 220 of the shoe 200 according to one embodiment of the present disclosure may perform a function of fixing and supporting a user's foot. In one embodiment, the bottom plate part 220 may be formed integrally with the protection part 210 and may be formed of the same material as that of the protection part 210.

The midsole 230 of the shoe 200 according to one embodiment of the present disclosure may be disposed below the bottom plate part 220 to perform a function of absorbing shocks when walking. According to one embodiment of the present disclosure, the midsole 230 may be made of a material softer than that of the bottom plate part 220 to perform functions of shock absorption and load dispersion. The midsole 230 may be made of, for example, ethylene vinyl acetate (EVA), urethane, polyurethane (PU), foamed sponge, rubber, or the like.

In one embodiment, at least one protrusion may be formed at at least a portion of the midsole 230 to protrude upward. At least one hole may be formed in the bottom plate part 220 so as to correspond to the at least one protrusion. The at least one protrusion of the midsole 230 may be inserted into the at least one hole of the bottom plate part 220 such that they are coupled to each other. The at least one protrusion of the midsole 230 may be formed at a portion corresponding to a heel of the shoe 200. With this configuration, it is possible to alleviate shocks exerted to the heel portion when walking.

Further, surfaces at which the midsole 230 and the bottom plate part 220 face each other, that is, an upper surface of the midsole 230 and a lower surface of the bottom plate part 220, may be firmly bonded to each other by an adhesive.

The toecap 240 of the shoe 200 according to one embodiment of the present disclosure may be formed to surround user's toes from the above and may perform a function of protecting the toes from external shocks. The toecap 240 may be configured to be attachable to and detachable from an upper side of a front portion of the bottom plate part 220. If necessary, the toecap 240 may be separated from the bottom plate part 220.

According to one embodiment of the present disclosure, the sole 100 may be detachably coupled to the shoe 200. Specifically, the midsole 230 of the shoe 200 may be inserted into a concave portion 110 formed in an upper surface of the sole 100 such that the shoe 200 and the sole 100 are coupled to each other. In one embodiment, the sole 100 is configured to surround a lower portion of the midsole 230 and to be attachable to and detachable from the midsole 230.

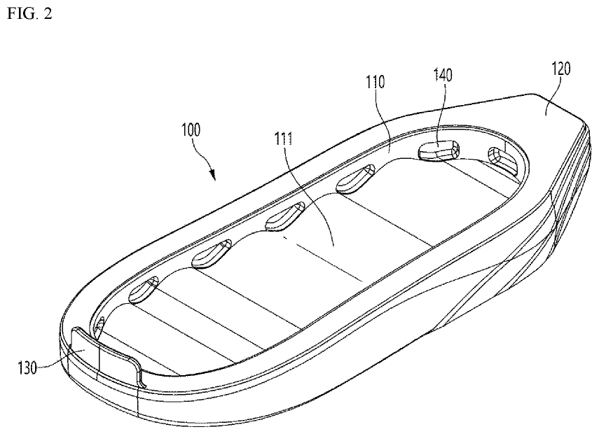

FIG. 2 shows a sole according to one embodiment of the present disclosure.

Referring to FIG. 2, the sole 100 includes the concave portion 110 formed in the upper surface thereof and into which the midsole 230 of the shoe 200 is inserted. As described above, the midsole 230 of the shoe 200 is inserted into the concave portion 110 of the sole 100 such that the midsole 230 is mounted on the sole 100.

According to one embodiment of the present disclosure, an inner sidewall of the concave portion 110 of the sole 100 may have a coupling portion to be coupled to an outer circumferential surface of the midsole 230. In one embodiment, a plurality of coupling protrusions 231 may be formed on the outer circumferential surface of the midsole 230, and a plurality of coupling holes 140 may be formed in the sole 100 at positions corresponding to the plurality of coupling protrusions 231 of the midsole 230.

According to one embodiment of the present disclosure, bent portions may be formed on the upper surface of the sole 100 at the concave portion 110 along a longitudinal direction of the sole 100. In one embodiment, first bent portions 232 may be formed on the lower surface of the midsole 230 of the shoe 200, and second bent portions 111 may be formed on the upper surface of the sole 100 at the concave portion 110 to have shapes corresponding to the first bent portions 232 of the midsole 230. With this configuration, when the midsole 230 is inserted into the sole 100, the coupling protrusions 231 of the midsole 230 are fitted into the respective coupling holes 140 of the sole 100 and simultaneously the first bent portions 232 of the midsole 230 slidably move along the second bent portions 111 of the sole 100 so that they are engaged with each other.

According to one embodiment of the present disclosure, the sole 100 may be made of a flexible material, for example, ethylene vinyl acetate (EVA), urethane, polyurethane (PU), foamed sponge, rubber, or the like.

As described above, the midsole 230 and the sole 100 are made of a soft material with flexibility. Thus, when the midsole 230 is inserted into the sole 100, the coupling protrusions 231 of the midsole 230 may be inserted into the respective coupling holes 140 formed in the positions corresponding to the coupling protrusions 231 while the coupling protrusions 231 move inward of the concave portion 110. The shape and number of the coupling protrusions 231 and the coupling holes 140 are not limited to those illustrated herein but may be variably changed in consideration of retention of the engagement, detachability, or the like.

According to one embodiment of the present disclosure, the sole 100 may be configured to be made of a material relatively softer than the midsole 230. Accordingly, when the midsole 230 is coupled to the sole 100, the sole 100 may widen so that the midsole 230 is easily inserted into the sole 100. When the sole 100 is separated from the midsole 230, it may be flexibly bent to be easily separated from the midsole 230. In another embodiment, the midsole 230 and the sole 100 may be made of the same material.

According to one embodiment of the present disclosure, the sole 100 may have a protruding portion 120. The protruding portion 120 may be formed in a portion corresponding to a heel of the shoe 200 in a case in which the sole 100 is mounted on the shoe 200, and may be formed to protrude backward from the heel of the shoe 200. Due to the protruding portion 120, the user may separate the sole 100 from the midsole 230 in a simplified manner without having to use his/her hand(s). Details thereof will be described later.

According to one embodiment of the present disclosure, the sole 100 may include a doorsill 130. The doorsill 130 may perform a function of easily aligning a position of the shoe 200 when the sole 100 is coupled to the shoe 200. In one embodiment, the doorsill 130 may be formed in a portion corresponding to a front side of the shoe 200 (for example, a toe) in the case in which the sole 100 is mounted on the shoe 200, and may be configured to protrude upward compared to other portions in the upper surface of the sole 100. Thus, the midsole 230 may be inserted into the concave portion 110 in a state in which the front side of the shoe 200 comes into contact with the doorsill 130. Accordingly, the user may easily couple the shoe 200 to the sole 100 in a standing state.

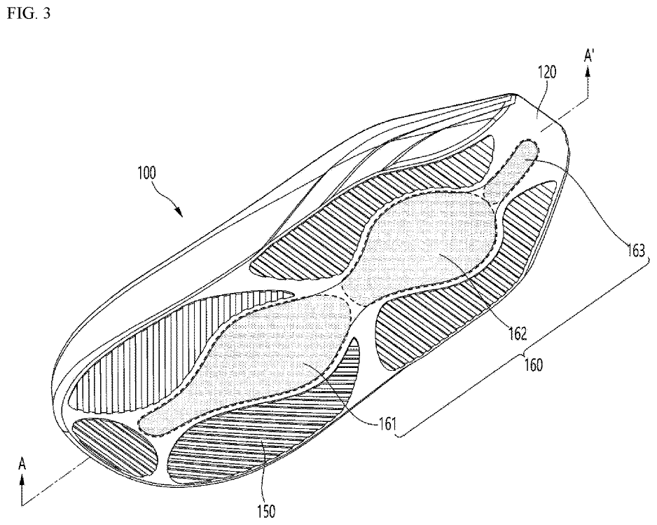

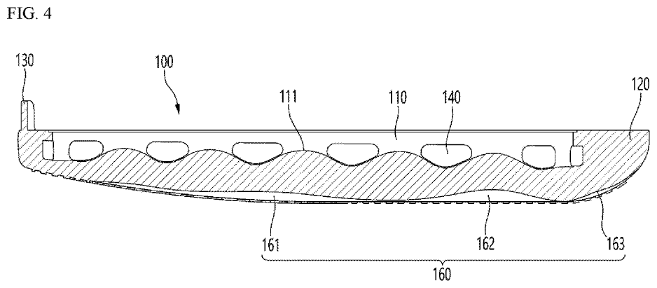

FIG. 3 shows a bottom surface of a sole according to one embodiment of the present disclosure. FIG. 4 shows a vertical cross-section of a sole according to one embodiment of the present disclosure. A structure of the bottom surface of the sole 100 according to one embodiment of the present disclosure will be described later in detail with reference to FIGS. 3 and 4.

According to one embodiment of the present disclosure, an anti-slip member 150 may be formed on the bottom surface of the sole 100 to prevent slippage. By the anti-slip member 150 of the sole 100, a road-hugging force of the shoe 200 when walking may be enhanced.

In one embodiment, a plurality of anti-slip members 150 having different directional patterns may be formed on the bottom surface of the sole 100 to prevent slippage in different directions. Further, the plurality of anti-slip members 150 may be formed to be spaced apart from each other at predetermined positions. This makes it possible to suppress the sole 100 from slipping due to hydroplane. Specifically, assuming that the user steps on standing water or the like on the ground, in a case in which the bottom surface is formed to have a simple flat structure or the anti-slip member 150 is formed to have a simplified structure, load may be distributed over the entire bottom surface so that the water does not spread quickly between the ground and the sole 100. This may cause the hydroplane. As a result, the road-hugging force of the shoe 200 may be reduced, which may cause the user to slip and get injured. In contrast, when the sole 100 according to one embodiment of the present disclosure is used, the load may be concentrated to each of the anti-slip members 150 so that the water below the anti-slip members 150 may be quickly drained via gaps between the anti-slip members 150. This may prevent the occurrence of the hydroplane.

The sole 100 according to one embodiment of the present disclosure may have at least one groove portion formed to be depressed upward from the bottom surface. In the illustrated embodiment, three groove portions 161, 162 and 163 may be formed in the bottom surface of the sole 100.

Among the three groove portions formed in the bottom surface of the sole 100, the groove portion 163 may be formed to open backward and be in communication with the outside in a state in which the bottom surface of the sole 100 is in contact with the ground. With this configuration, even if the sole 100 is used on a flat and smooth road surface, it is possible to prevent a situation in which a vacuum is formed under the sole 100 and the sole 100 is suctioned to the ground. Further, even if the user steps on the standing water or the like on the ground, the water in other groove portions may be drained via the groove portion 163, which makes it possible to prevent the occurrence of the hydroplane.

Further, referring to FIG. 4, the bottom surface of the sole 100 may have a round shape along a longitudinal direction when viewed from a lateral side (that is, a direction from the toe to the heel). In a case in which a sole with a flat bottom surface is applied to a splint-type shoe, stress may be applied to the user's knee joints or the like when walking. The sole 100 according to one embodiment of the present disclosure may reduce such stress.

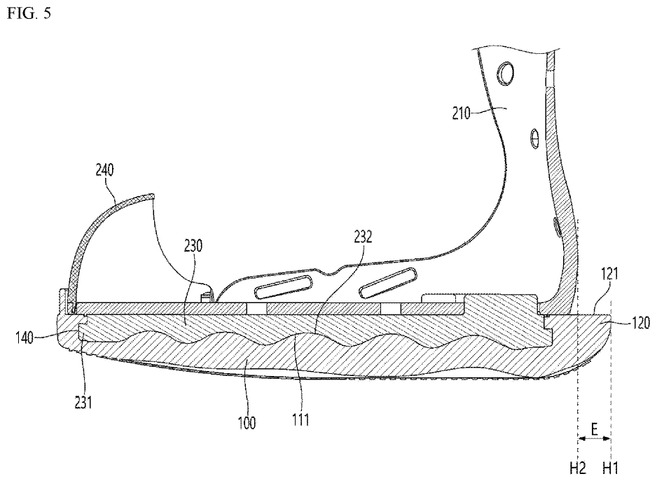

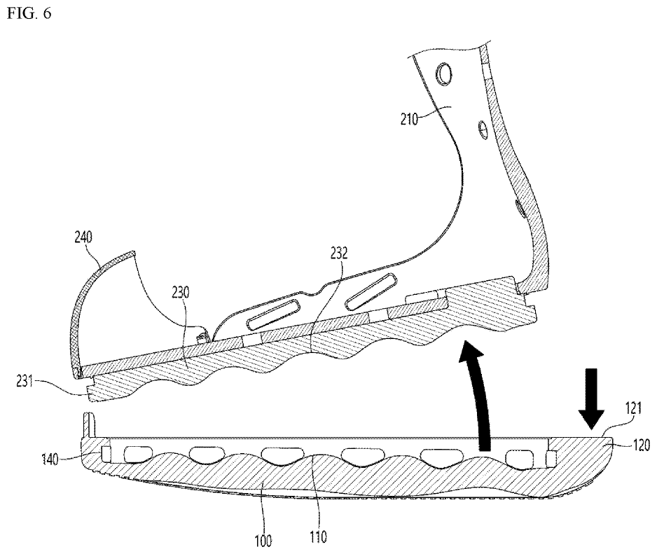

FIG. 5 shows a state in which a sole according to one embodiment of the present disclosure is coupled to a shoe. FIG. 6 shows a state in which a sole according to one embodiment of the present disclosure is separated from a shoe.

Referring to FIG. 5, the protruding portion 120 of the sole 100 according to one embodiment of the present disclosure may be formed at a position corresponding to the heel of the shoe 200 in the case in which the sole 100 is mounted on the shoe 200, and may be formed to protrude backward from the heel of the shoe 200. For example, the protruding portion 120 may be formed by protruding a rear end Hl of the sole 100 backward. The rear end Hl is protruded backward by a distance E from a heel portion H2 of the shoe 200. Thus, an upper portion 121 of the protruding portion 120 is exposed backward of the shoe 200.

Referring to FIG. 6, the user may press the upper portion 121 of the protruding portion 120 with the other foot to fix the sole 100 with respect to the ground, and lift up his/her foot to separate the sole 100 from the midsole 230 of the shoe 200. As described above, since the sole 100 and/or the midsole 230 are made of the elastic material such as EVA, urethane, PU, foamed sponge, rubber, or the like, the coupling protrusions 231 of the midsole 230 may be easily separated from the respective coupling holes 140 of the sole 100.

As described above, when the user tries to mount the sole 100 to the shoe 200 (for example, when the user tries to move from indoors to outdoors), the user presses the shoe 200 he/she is wearing against the sole 100 from the above in a state in which the sole 100 is located at a proper position on the floor. Thus, the midsole 230 of the shoe 200 is aligned with the sole 100 in conformity to the bent portions formed in the concave portions 110 of the sole 100. In this way, the midsole 230 is seated on and coupled to the sole 100. In this case, the shoe 200 and the sole 100 may be positioned in a further convenient manner by the doorsill 130. Even if the user accidentally places the sole 100 at an unintended position on the floor, he/she may push the doorsill 130 with the shoe 200 to adjust the position of the sole 100.

In addition, when the user tries to separate the sole 100 from the shoe 200 (for example, when the user moves from outdoors to indoors), the user may simply separate the midsole 230 from the sole 100 by using the protruding portion 120 while fixing the sole 100 to the ground. In this case, since the groove portions are formed in the bottom surface of the sole 100, the occurrence of vacuum phenomenon may be prevented. This makes it possible to prevent the sole 100 from being suctioned to the ground.

Accordingly, the user does not need to bend his/her waist or legs when moving from indoors to outdoors or vice versa, and may separate the sole 100 from the shoe 200 in a convenient manner. Thus, even a patient with limited mobility may use the sole 100 in a convenient manner. Further, since there is no need to fully put on or take off the shoe 200, it is possible to continuously maintain the splint effect. Further, the user may separate the sole 100 from the shoe 200 without having to use his/her hand(s), which enables hygienic usage.

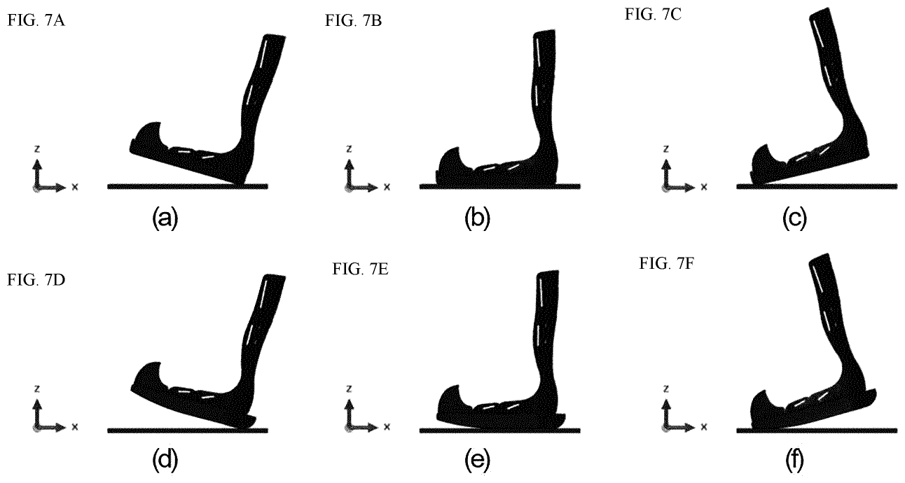

FIGS. 7A to 7F illustrate walking states when the user walks with soles according to Example of the present disclosure and Comparative example. Specifically, FIGS. 7A to 7C illustrate the walking states when the user walks using a sole with a flat bottom surface according to Comparative example, and FIG. 7D to 7F illustrate the walking states when the user walks using a sole with a rounded bottom surface according to Example embodiment of the present disclosure. Further, FIGS. 7A and 7D illustrate states in which a heel portion comes into contact with the ground when the user's foot starts to come into contact with the ground. FIGS. 7B and 7E illustrate states in which a middle portion of the bottom surface comes into contact with the ground. FIGS. 7C and 7F illustrate states before the user's foot leaves the ground, that is, states in which only a front portion of the sole comes into contact with the ground.

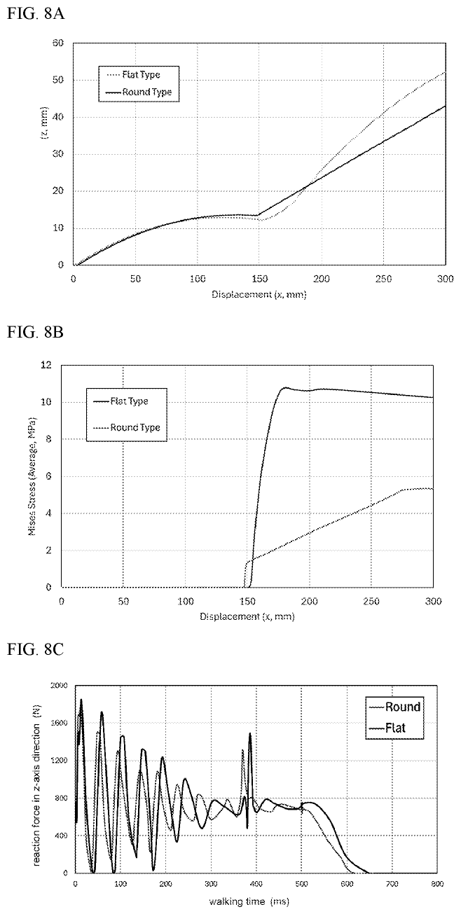

FIGS. 8A and 8C are graphs illustrating walking simulation results when the user walks using soles according to Example embodiment of the present disclosure and Comparative example, which illustrate results obtained by simulating displacement of knee joints, stress applied to an ankle portion, reaction force, and the like in the walking states illustrated in FIGS. 7A to 7F. In FIGS. 8A to 8C, there are illustrated simulation results in a case of walking using the sole made of EVA material (with elastic modulus of 70 MPa) on a bottom surface formed of an iron material (with elastic modulus of 2.07 GPa).

FIG. 8A is the graph illustrating the result obtained by simulating the displacement of knee joint, in which the horizontal axis represents the displacement of the knee joint with respect to a walking direction, and the vertical axis represents a vertical displacement of the knee joint with respect to the ground. Referring to FIG. 8A, there was no significant difference in the vertical displacement until the user walked in the walking direction by 150 mm, but the vertical displacement in the vertical direction after the user walked by 150 mm was decreased by 17.5% in Example of the present disclosure compared to Comparative example.

FIG. 8B is the graph illustrating the result obtained by simulating an average Von Mises stress occurring at the ankle portion, in which the horizontal axis represents the displacement of the knee joint with respect to the walking direction, and the vertical axis represents the average Von Mises stress occurring at the ankle portion. Referring to FIG. 8B, the maximum value of the average Von Mises stress when using the sole according to Example of the present disclosure was decreased by 50.1% compared to Comparative example.

As can be seen from FIGS. 8A and 8B, when using the bottom surface with the round shape of the sole according to Example of the present disclosure, the displacement of the knee joint was decreased as compared to the case of using the sole with the flat bottom surface. Further, the load applied to the ankle portion was also decreased. Thus, the user may walk comfortably and stably.

FIG. 8C is the graph illustrating the result obtained by simulating a vertical reaction force after the load applied to the sole is removed, in which the horizontal axis represents a walking time, and the vertical axis represents the vertical reaction force applied to the sole from the ground. The simulation on the vertical reaction force was conducted by applying a weight and an initial speed for a period of 500 ms and checking a time taken for the user's foot to leave the floor when the user's weight is transferred toward the other foot. In this case, it can be seen that the foot leaved the bottom surface when a magnitude of the vertical reaction force becomes zero.

Referring to FIG. 8C, it was found that after the load is removed, the time taken for the vertical reaction force to become zero is 49.63 ms in Comparative example, and is 39.48 ms in Example of the present disclosure. That is, it was found that, when using the sole with the rounded bottom surface, the time taken for the user's foot to leave the floor is reduced by 20.5%. This teaches that, by using the sole according to Example of the present disclosure, the walking speed of the user and walking convenience are improved as compared to Comparative example.

As described above, the sole according to one embodiment of the present disclosure is configured to have the rounded bottom surface. In particular, in the case in which the sole is applied to the splint-type shoe (so-called leg protector), it is possible to reduce discomfort to be applied to the user when walking, ensure stable walking, and increase the walk speed.

Although the present disclosure has been described above in terms of specific items such as detailed constituent elements as well as the limited exemplary embodiments, they are merely provided to help more general understanding of the present disclosure, and the present disclosure is not limited to the above exemplary embodiments. Various modifications and changes could have been realized by those skilled in the art to which the present disclosure pertains from the above description.

Therefore, the spirit of the present disclosure need not to be limited to the above-described exemplary embodiments, and in addition to the appended claims to be described below, and all ranges equivalent to or changed from these claims need to be said to belong to the scope and spirit of the present disclosure.

What is claimed is:

1. A detachable sole on shoes, comprising:
a concave portion formed in an upper surface sole and configured such that a midsole of a shoe is inserted thereinto;
a protruding portion formed at a portion corresponding to a heel of the shoe when the detachable sole is mounted on the shoe and configured to protrude further backward than the heel of the shoe; and
at least one groove portion formed to have a concave shape facing upward in a bottom surface of the sole.

2. The detachable sole of claim 1, wherein the bottom surface has a round shape along a longitudinal direction when viewed from a lateral side.

3. The detachable sole of claim 1, wherein the at least one groove portion is formed to open backward and communicate with an outside in a state in which the bottom surface is brought into contact with a ground.

4. The detachable sole of claim 1, wherein an inner sidewall of the concave portion includes a coupling portion configured to be coupled to an outer circumferential surface of the midsole.

5. The detachable sole of claim 4, wherein the coupling portion includes a plurality of coupling holes formed along a periphery of the inner sidewall of the concave portion, the plurality of coupling holes being configured to be coupled to a plurality of coupling protrusions formed on an outer circumferential surface of the midsole, respectively.

6. The detachable sole of claim 1, wherein an upper surface of the concave portion includes bent portions formed along a longitudinal direction.

7. A shoe comprising:
a protection part formed to surround at least a portion of a leg of a user;
a bottom plate part connected to the protection part and formed to support a foot of the user;
a midsole provided below the bottom plate part; and
a sole configured to be attachable to and detachable from the midsole,
wherein the sole includes:
a concave portion formed in an upper surface of the sole and configured such that the midsole is inserted thereinto;
a protruding portion formed at a portion corresponding to a heel of the shoe when the sole is mounted on the shoe and configured to protrude further backward than the heel of the shoe; and
at least one groove portion formed to have a concave shape facing upward in a bottom surface of the sole.
